(19) United States
(12) Patent Application Publication
(10) Pub. No.: US 2007/0043420 A1
(43) Pub. Date: Feb. 22, 2007

(54) Apparatus and method for stent-graft release using a cap

(75) Inventor: Timothy W. Lostetter, Cooper City, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/207,171

(22) Filed: Aug. 17, 2005

(51) Int. Cl. A61F 2/06

(52) U.S. Cl. 623/1.11

(57) ABSTRACT

A stent-graft deployment system (10) can include a stent-graft (15), a catheter (21) having a flexible catheter tip (12) attached to a catheter shaft of the catheter, a retractable primary sheath (20) containing the stent-graft in a first constrained small diameter configuration around the catheter shaft near the flexible tip, and a pushrod (18) having a cup (16) containing part of or substantially all of a distal spring at the end thereof for retaining a distal end of the stent graft in a constrained position. The cup plunger moves coaxially in relation to the catheter and the retractable primary sheath. The stent-graft deployment system can further include a release plate (17) coupled to the catheter and with the release plate held stationary the cup moves coaxially relative to the release plate acting as a barrier so as the cup retracts the proximal end of the stent graft beyond an outer edge of the cup is exposed to release the stent-graft from the constrained position to enable stent-graft deployment.

### FIELD OF THE INVENTION

This invention relates generally to medical devices and procedures, and more particularly to a method and system of deploying a stent-graft in a vascular system.

### BACKGROUND OF THE INVENTION

Prostheses for implantation in blood vessels or other similar organs of the living body are, in general, well known in the medical art. For example, prosthetic vascular grafts formed of biocompatible materials (e.g., Dacron or expanded, porous polytetrafluoroethylene (PTFE) tubing) have been employed to replace or bypass damaged or occluded natural blood vessels. A graft material supported by framework is known as a stent-graft or endoluminal graft. In general, the use of stent-grafts for treatment or isolation of vascular aneurysms and vessel walls which have been thinned or thickened by disease (endoluminal repair or exclusion) are well known. Many stent-grafts, are “self-expanding”, i.e., inserted into the vascular system in a compressed or contracted state, and permitted to expand upon removal of a restraint. Self-expanding stent-grafts typically employ a wire or tube configured (e.g. bent or cut) to provide an outward radial force and employ a suitable elastic material such as stainless steel or Nitinol (nickel-titanium). Nitinol may additionally employ shape memory properties. The self-expanding stent-graft is typically configured in a tubular shape of a slightly greater diameter than the diameter of the blood vessel in which the stent-graft is intended to be used. In general, rather than inserting in a traumatic and invasive manner, stents and stent-grafts are preferably deployed through a less invasive intraluminal delivery, i.e., cutting through the skin to access a lumen or vasculature or percutaneously via successive dilatation, at a convenient (and less traumatic) entry point, and routing the stent-graft through the lumen to the site where the prosthesis is to be deployed.

Intraluminal deployment is typically effected using a delivery catheter with coaxial inner (plunger) and outer (sheath) tubes arranged for relative axial movement. The stent is compressed and disposed within the distal end of an outer catheter tube in front of an inner tube. The catheter is then maneuvered, typically routed though a lumen (e.g., vessel), until the end of the catheter (and the stent-graft) is positioned in the vicinity of the intended treatment site. The innertube is then held stationary while the outertube of the delivery catheter is withdrawn. The inner tube prevents the stent-graft from being withdrawn with the outer tube. As the outer tube is withdrawn, the stent-graft radially expands so that at least a portion of it is in substantially conforming surface contact with a portion of the interior of the lumen e.g., blood vessel wall.

Some stent-graft deployment systems use a disc shaped or shallow cup plunger configuration to act as a barrier at a distal end (position relative to its deployed location in the vasculature from the heart) of a stent-graft to prevent movement of the stent graft relative to the catheter center member as and until an outer tube or sheath is withdrawn, causing the springs on the distal end of the stent-graft to deploy or release upon sheath retraction without much control by the physician. A shallow cup plunger provides no extra control of the radial deployment of the distal end of the stent graft.

In instances where the springs at the proximal end of the stent graft are held captured to the catheter to permit repositioning, the unconstrained release of the distal end of the stent graft limits how far the outer tube or sheath can be retracted before repositioning cannot be done. So once the distal end of the stent-graft is deployed, the physician loses the ability to manipulate the stent-graft axially, radially, or tortially or in a twisting manner. Thus, existing cup plunger assemblies fail to encapsulate (hold) the distal end of stent-grafts before, during, and after deployment of a sheath and further fail to contribute to the controlled deployment of the stent-graft after an outer sheath is withdrawn in a delivery configuration where the proximal end is also held constrained, or had been held by another mechanism prior to deployment of the distal end.

### SUMMARY OF THE INVENTION

In a first embodiment according to the present invention, a stent-graft release mechanism can include a catheter, a coaxial inner tube having a cup at a distal end where the coaxial inner tube is placed about the catheter, a release plate affixed to the catheter, and a mechanism for axially moving the release plate relative to the cup.

In a second embodiment, a stent-graft deployment system can include a stent-graft, a catheter having a flexible catheter tip attached to a catheter shaft of the catheter, a retractable primary sheath containing the stent-graft in a first constrained small diameter configuration around said catheter shaft near said flexible tip, and a cup plunger having a cup operatively coupled at the end thereof for retaining a distal end of the stent graft in a constrained position, where the cup plunger moves coaxially in relation to the catheter and the retractable primary sheath. The stent-graft deployment system can further include a release plate coupled to the catheter, wherein the release plate moves coaxially relative to the cup for pushing the distal end of the stent graft beyond an outer edge of the cup in order to release the stent-graft from the constrained position to enable stent-graft deployment.

In a third embodiment, a method of deploying a stent-graft using a stent-graft deployment system having a stent-graft release mechanism and a retractable primary sheath, includes the steps of loading the stent-graft deployment system with a stent-graft, where the distal end of the stent-graft is retained within a cup of the stent-graft release mechanism and tracking the stent-graft deployment system over a guide wire to a location before a target area. The method can further include the step of retracting the primary sheath to expose at least a proximal portion of the stent-graft and moving a release plate from within a lower portion of the cup to beyond a distal edge of the cup to at least partially deploy the stent-graft in the target area.

In the third embodiment, the method can further include the step of retaining apexes of Nitinol springs of the distal end of the stent-graft within the cup before deployment. The catheter can be coupled to the release plate and the step of moving the release plate can include the step of rotating a luer that coaxially moves the catheter relative to the cup. A secondary sheath can move axially within the primary sheath wherein the method can further include the step of moving the stent-graft to a location within a target area while the primary sheath is retracted as the secondary sheath is exposed. The step of moving the release plate can include the step of axially moving the release plate relative to the cup. Additionally, the method can further include the step of releasing the stent-graft from the delivery system after moving the release plate beyond an edge of the cup.

### BRIEF DESCRIPTION OF THE DRAWINGS

FIG. 1 is a plan view of a stent-graft deployment system without a stent-graft in accordance with the present invention.

FIG. 2 is a close up schematic plan view of the deployment system of FIG. 1 having a loaded stent-graft.

FIG. 3 illustrates the stent-graft deployment system of FIG. 1 with a primary sheath partially retracted to expose a secondary sheath (in dashed lines).

FIG. 4 illustrates the stent-graft deployment system of FIG. 1 with the primary sheath retracted and the secondary sheath partially retracted.

FIG. 5 illustrates the stent-graft deployment system of FIG. 1 with the primary sheath retracted with the secondary sheath almost completely retracted and the distal end of the stent-graft constrained by the cup in accordance with the invention.

FIG. 6 illustrates the stent-graft deployment system of FIG. 1 with the secondary sheath completely retracted and the stent-graft fully deployed using a stent-graft release mechanism in accordance with the present invention.

FIG. 6A illustrates the stent-graft deployment system of FIG. 6 with the stent-graft partially deployed using an alternative arrangement stent-graft release mechanism in accordance with the present invention.

FIG. 6B illustrates the stent-graft deployment system of FIG. 6A with the stent-graft fully deployed using the alternative arrangement stent-graft release mechanism.

FIG. 6C is a close up schematic plan view of a portion of a stent-graft deployment delivery system with a plurality of proximal springs constrained within a cap of the alternative arrangement in accordance with an embodiment of the present invention.

FIG. 6D illustrates the deployment delivery system of FIG. 6C with the plurality of proximal springs released from under the cap.

FIG. 7 is a close-up view of the cup plunger and release plate before deployment in accordance with the present invention.

FIG. 8 is a close-up view of the cup plunger and release plate after deployment in accordance with the present invention.

FIG. 9 is a close-up view of the cup plunger and release plate and further illustrating the stent-graft before deployment in accordance with the present invention.

FIG. 10 is a close-up view of the cup plunger and release plate and further illustrating the stent-graft after deployment in accordance with the present invention.

FIG. 11 is a flow chart illustrating the steps of a method in accordance with the present invention.

### DETAILED DESCRIPTION

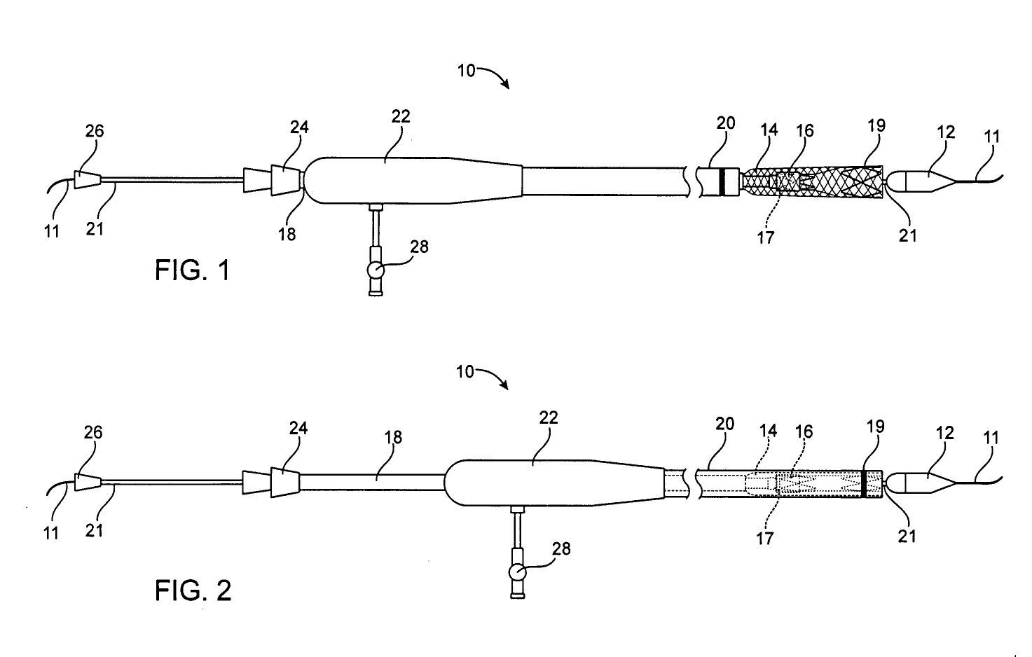

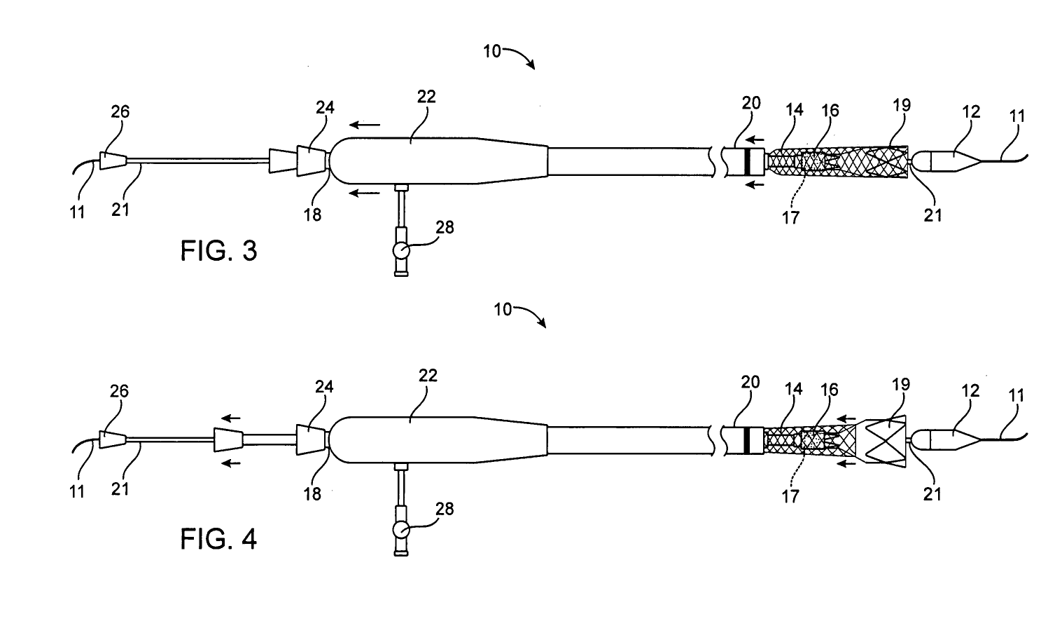

FIGS. 1-3 show portions of a stent-graft deployment system 10. FIG. 1 illustrates the system 10 without a stent-graft while FIGS. 2 and 3 show additional views of the deployment system which is loaded with a stent-graft 15 within a secondary sheath 14 (such an arrangement is described in U.S. Patent Publication No. 2005/0038495, incorporated herein by reference) and further includes a stent-graft release mechanism as will be further detailed below. This system could also deploy a stent alone or some other form of endoprosthesis. The subsequent use of “stent-graft” herein should be understood to include other forms of endoprosthesis. Ideally, the stent-graft deployment system 10 comprises a tapered tip 12 that is flexible and able to provide trackability in tight and tortuous vessels. Other tip shapes such as bullet-shaped tips could also be used.

The system 10 includes a primary sheath 20 (preferably made of a semi-rigid material such as PTFE) initially covering an optional secondary sheath 14 (preferably made of woven polyethylene terephthalate (PET)). The secondary sheath 14 can be more flexible than the retractable primary sheath 20. The deployment system 10 is able to separately retract the primary and secondary sheaths.

The primary sheath should have enough stiffness and column strength to provide adequate pushability as the system 10 tracks through small diameter vessels that tend to conform to the shape of the delivery system. The secondary sheath utilizes its greater flexibility (at the expense of column strength) to improve trackability and advancement, in vessels with larger diameters that do not tend to conform to the shape of the delivery system, particularly through areas having tight radiuses. So, where prior deployment systems utilizing just a semi-rigid primary sheath were prone to kinking while tracking through an area with a tight radius, the secondary sheath of the present invention avoids kinking and easily adapts to the shape of the vessel which reduces advancement force while tracking through the vessels with tight curves. The greater flexibility and potential for larger sheath diameter in the secondary sheath can greatly reduce resistance to deploy the stent graft in areas with tight curves.

The deployment system 10 also includes a stent-graft 15 initially retained within the secondary sheath 14. As described herein, the stent-graft 15 is a self-expanding, Nitinol/Dacron stent-graft system designed for endovascular exclusion of Thoracic Aortic Aneurisms (TAA). The deployment system 10 includes a cup 16 and release plate 17 as shown in FIGS. 1-10 that serves to retain the stent-graft 15 in place during deployment and further serves as part of a stent-graft release mechanism. The cup 16 is preferably a deep cup that encapsulates a large longitudinal length of if not the full longitudinal length of a spring at a distal end of the stent-graft (as shown in FIG. 9) and remains encapsulated while the primary sheath 20 extends over the stent-graft and after the sheath is retracted. The cup 16 can have a depth of at least 0.25 inches, although shallower or deeper depths are certainly contemplated herein. In this regard, most embodiments can include cups having depths that range from about 25% of the longitudinal length of the spring (in a compressed form at the distal end of the stent-graft) up to 100% of the length. It is expected that cup depths of 40%, 45%, 50%, 66⅔%, 75%, and 100% of the compressed axial length of the stent spring at the distal end of the stent graft would be engaged or enveloped by the surrounding cylindrical walls of the cup plunger. Of course, other embodiments can have cup depths with lesser or greater percentages than described above. A handle or a hub 22 is fixed to the primary sheath 20, a second handle or hub (24) near a proximal end of the stent-graft deployment system 10 is fixed to the secondary sheath via 14 a plunger or pushrod 18, and a catheter shaft 21 is connected to a shaft handle 26 and aids the advancement of the system 10 and acts as a deployment means as will be made apparent below. In addition, the deployment system 10 shown can include a guide wire 11, a flush port 28, and a radiopaque marker 19 allowing for accurate positioning of the delivery system prior to deployment of the stent-graft in the proximal position.

FIG. 2 illustrates the stent-graft deployment system 10 with the primary sheath 20 covering the secondary sheath 14 (wherein flexible secondary sheath 14 is arranged within the semi-rigid sheath 20 when the semi-rigid sheath 20 is in a non-retracted position), FIGS. 1, 3, and 4-6 illustrate the primary sheath 20 retracted and exposing the secondary sheath 14. With respect to FIGS. 4-6 and FIGS. 9-10, the stent-graft deployment system 10 is shown in various stages as it advances over a guide wire (not shown) and the stent-graft is deployed. FIGS. 4-6 and 9-10, in particular, illustrate the stent-graft deployment system 10 as it would operate or function outside or apart from the body. Note that this embodiment is ideally suited for tracking over a guide wire within a body and particularly through a target area (vessel) having a tight curvature or radius.

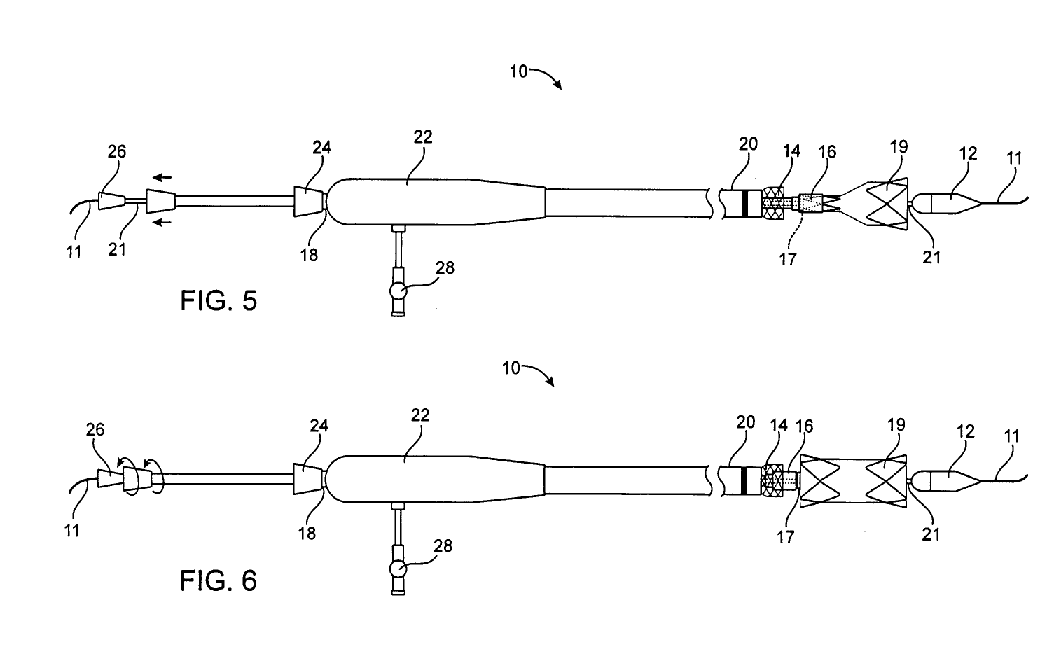

As shown in FIGS. 3-5, the stent-graft 15 is constrained by the flexible secondary sheath 14 as well as by the cup 16. The cup constrains the distal end of the stent graft even when the stent-graft 15 is nearly deployed as shown in FIG. 5. The embodiment shown in FIGS. 2-6 further illustrates the handle or hub 22 coupled to the semi-rigid sheath 20 serving as a first arrangement for retracting the semi-rigid sheath 20 and exposing the flexible secondary sheath 14 as well as an inner tube 18 coupled to the flexible secondary sheath 14 serving as a second arrangement for retracting the flexible secondary sheath and enabling unconstrained portions of the stent-graft to expand. It should be noted that the exposed portion of the flexible secondary sheath 14 could have a diameter larger than the semi-rigid primary sheath 20 that surrounded the flexible secondary sheath 14 previously. The larger diameter of the exposed portion of the flexible secondary sheath 14 is a contributory factor in reducing the force needed to retract the secondary sheath. Once the flexible secondary sheath 14 is exposed, the end of stent-graft deployment system 10 beyond the semi-rigid sheath 20 has greater flexibility (than the portion of the system within the semi-rigid sheath 20) as it tracks across the guidewire. Once the secondary sheath 14 is exposed or outside the primary sheath, the system 10 can be advanced over the guide wire with a lower advancement force since the secondary sheath is designed to be quite flexible particularly in vessel areas with tight radiuses.

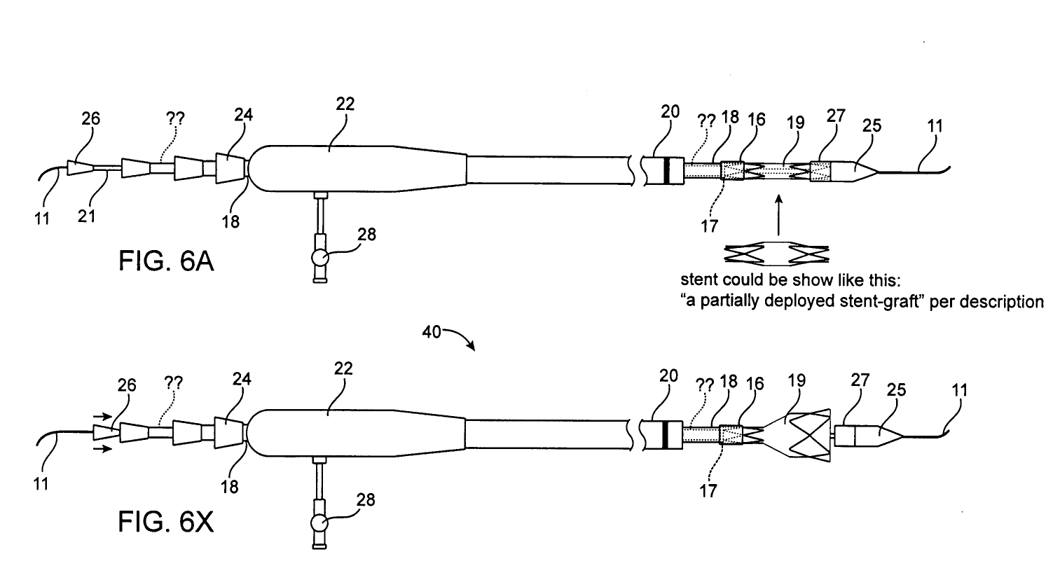

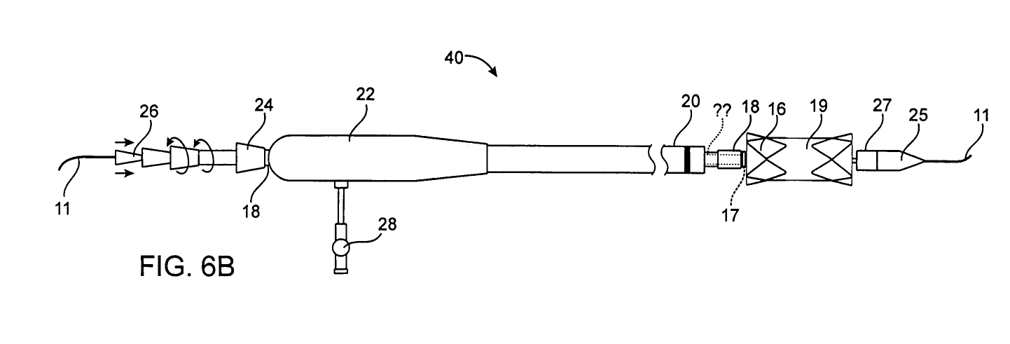

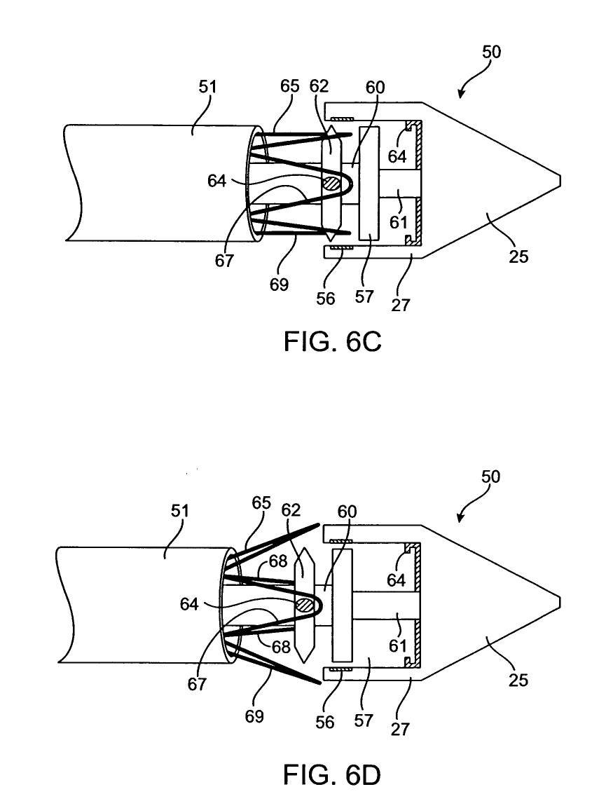

Referring to FIGS. 4-5, the primary sheath has been retracted and the secondary sheath is shown partially retracted with the stent-graft 15 being partially deployed. As the secondary sheath retracts, more and more of the stent-graft is deployed until the secondary sheath 14 is completely retracted and the stent-graft 15 is fully deployed as shown in FIG. 6.

Operationally, once the secondary sheath 14 is exposed as shown in FIG. 3, the stent-graft 15 can emerge out of the secondary sheath 14 by retracting the cup 16 coaxially relative to the catheter 21 as shown in FIGS. 4 and 5 in a controlled manner. This can be achieved by having handle 26 (for example a luer) operatively coupled to the cup 16 such that rotation of the handle 26 can incrementally move the release plate 17 relative to the cup 16. As shown in FIG. 6, as the cup 16 is retracted the release plate will nudge the stent-graft 15 into full deployment as cup 16 reaches nearly full retraction. It should be understood that the cup 16 does not necessarily need to go beyond the edge of the release plate to release the stent-graft 15 into full deployment. The cup 16 and release plate 17 provide a controlled deployment of the distal spring once the stent-graft is in place and further provide constant engagement of the distal stent-graft spring apexes during tracking of the system through the vessels of a patient.

In summary, the stent-graft release mechanism uses a cup that encapsulates the distal end of the stent-graft before, during and after deployment of the sheath and/or a proximal end of the stent-graft. The cup also serves as a positive engagement mechanism when the stent-graft is partially deployed in a flexible sheath such as the secondary sheath.

When using a proximal lock that retains the proximal end of the stent-graft during deployment, the stent-graft release mechanism provides additional maneuverability to a partially deployed stent-graft (See FIGS. 6A-D). The cup and release plate further enable a controlled deployment of the distal end of the stent-graft after withdrawal of a sheath. More specifically, to release the distal end of the stent-graft, the cup (16), which holds the spring apexes, can be retraced with respect to the catheter inner member (21) while the release plate (17) remains stationary which prevents the stent-graft from being dragged back with the cup. In this way, the cup and release plate enable the deployment of a stent-graft by acting as an engagement mechanism for the stent-graft so the sheath can be retraced over the stent-graft while in a partially expanded flexible sheath.

The embodiment of FIGS. 1-6 illustrates a stent-graft deployment system using a double sheath and a cup that is not necessarily fixed to a plunger or pushrod 18.

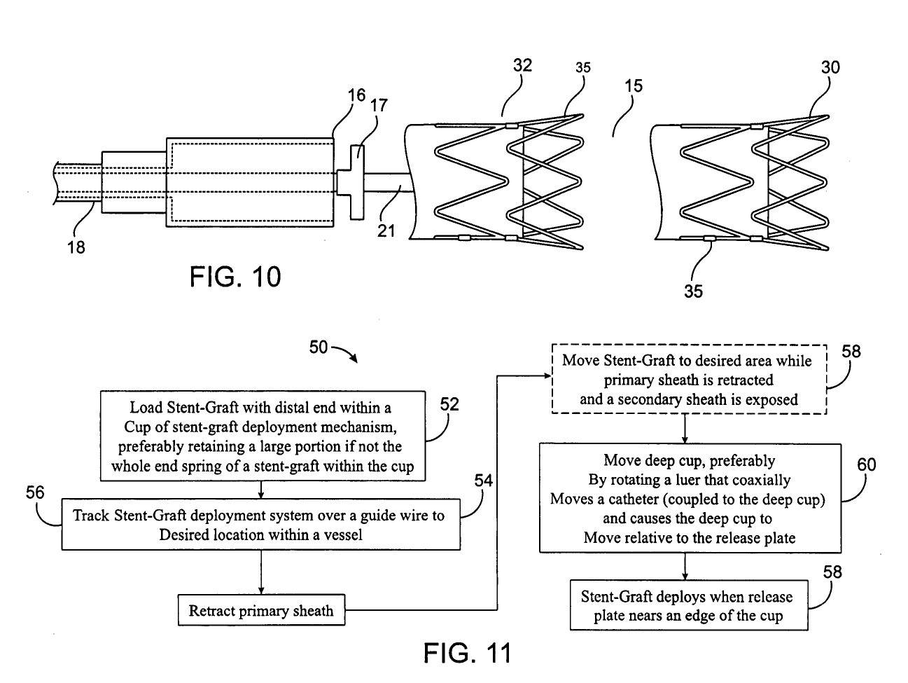

In an alternative embodiment where no secondary sheath is necessarily used as shown in FIGS. 6A and 6B, a stent-graft deployment system 40 includes a tapered tip 25 having retention means such as a cap 27 at a proximal end of the stent-graft and a cup 16 that can be attached to the end of a plunger or pushrod 18. Using the retention means (or a proximal lock that retains the proximal end of the stent-graft within the cap as illustrated and described below with regard to FIGS. 6C and 6D) in conjunction with the cup can give a partially deployed stent-graft as shown in FIG. 6A some maneuverability while traversing or attempting to place the stent-graft within a vessel. As shown in FIG. 6B, the stent-graft can be fully deployed once the release plate 17 urges the apexes 32 (as shown in FIG. 9) of the stent graft 15 out of the cup and once the proximal end of stent graft (and it's corresponding apexes 30 as shown in FIG. 10) is released from the retention means or cap 27.

Close up schematic plan views of another stent-graft deployment delivery system using an alternative arrangement stent-graft release mechanism 50 are shown in FIGS. 6C and 6D (further described in U.S. Patent Application Publication No. 2004/0093063, hereby incorporated by reference). Although mechanism 50 is primarily designed for a main stent-graft, this can also be used for deployment of a branch graft as well. The mechanism 50 of FIG. 6C illustrates a plurality of proximal springs 65, 67 and 69 (68 is hidden in this view) of the stent-graft 15 constrained within a cap or shroud portion (27) of a tip 25. The proximal springs 65, 67, 68, and 69 can be similar to the apexes 30 and 32 shown in FIG. 9. The cap or shroud portion 27 can be formed from a tube section made of a plastic like material for cap 27 which can further include a reinforcing support ring 56 made of a metal ring. FIG. 6D illustrates another close up view of the mechanism 50 with the plurality of proximal springs 65, 67, 69 and 68 released from under the cap 27 (by the cap having been moved upwards in FIG. 6D). The mechanism 50 includes an outer tube 60 within the retractable primary sheath (not shown) and within the stent-graft 15. The mechanism 50 can further include an inner tube 61 within the outer tube 60 having as a guidewire lumen therethrough. The inner tube 61 and the outer tube 60 preferably move axially relative to each other and can also move relative to the retractable primary sheath (not shown). The cap 27 is coupled to a distal end of the innertube 61 and is configured to retain at least a portion of a proximal end of the stent-graft 15 in a radially compressed configuration. A controlled relative axial movement between the outer tube 60 and the inner tube 61 releases the proximal end of the stent-graft (such as proximal springs) from the cap and from the radially compressed configuration.

The cap 27 can be formed from a shroud portion of the tapered tip 25 which is coupled at the distal end of the inner tube 61. Within the shroud portion (formed by the tubular body portion of the cap 27) preferably resides a back plate (disc) 57 coupled to a distal end of the outer tube 60 that serves as a proximal stop for the stent-graft 63 preventing movement in a proximal direction. The tubular body portion of the shroud portion may also include a support ring 56 near the proximal end of the tapered tip 25 to provide additional rigidity to the cap 27. Additionally, a proximal lock 62 is also coupled to a distal area of the outer tube 60. The proximal lock 62 preferably includes at least one or a plurality of ribs 64 that serves as an axial constraint for the stent-graft 63. The proximal end (or the proximal springs 65, 67, 68 and 69) of the stent-graft 63 cannot deploy until the ends of the proximal lock 62 clear the bottom end of the shroud portion of the tip.

A stent-graft can include a polyester or Dacron material (forming the graft material) sewn to a Nitinol support structure using polyester sutures. The Nitinol wire is used to form a skeletal structure that provides support, strength and stability to the stent-graft. The stent-graft can also have a support member on the proximal end of the stent-graft that is left mainly uncovered by the graft material. The uncovered portion will typically have a sinusoidal pattern with a predetermined number of apexes protruding up. The apexes form what is known as the proximal spring or springs of the stent-graft. As shown, the gap between the back plate 57 and the proximal lock 62 is preferably designed to hold the protruding apexes of the proximal spring. The apexes straddle the ribs 64 of the proximal lock 62 and remain trapped between the back plate 57 and the proximal lock until the relative movement between the outer tube 60 and the inner tube 61 exposes the gap and the proximal springs 65, 67, 68, and 69. In other words, the apexes cannot release from the ribs 64 on the proximal lock 62 while the apexes remain within the shroud portion of the cap 54. When the inner tube 61 and coupled tapered tip 25 are advanced forward exposing the proximal lock 62, the apexes of the proximal springs 65, 67, 68, 69 release from the respective ribs 64 of the proximal lock 62. The release results in the deployment of the proximal end of the stent-graft 15. Note that while the proximal springs 65, 67, 68, 69 remain in the gap and within the cap or shroud portion of the tapered tip 25, the proximal springs remain axially constrained as well as radially constrained. The support ring 56, usually made of metal, helps prevent the radial force of the proximal springs from distorting the shape of the tapered tip and particularly the shroud portion of the tapered tip.

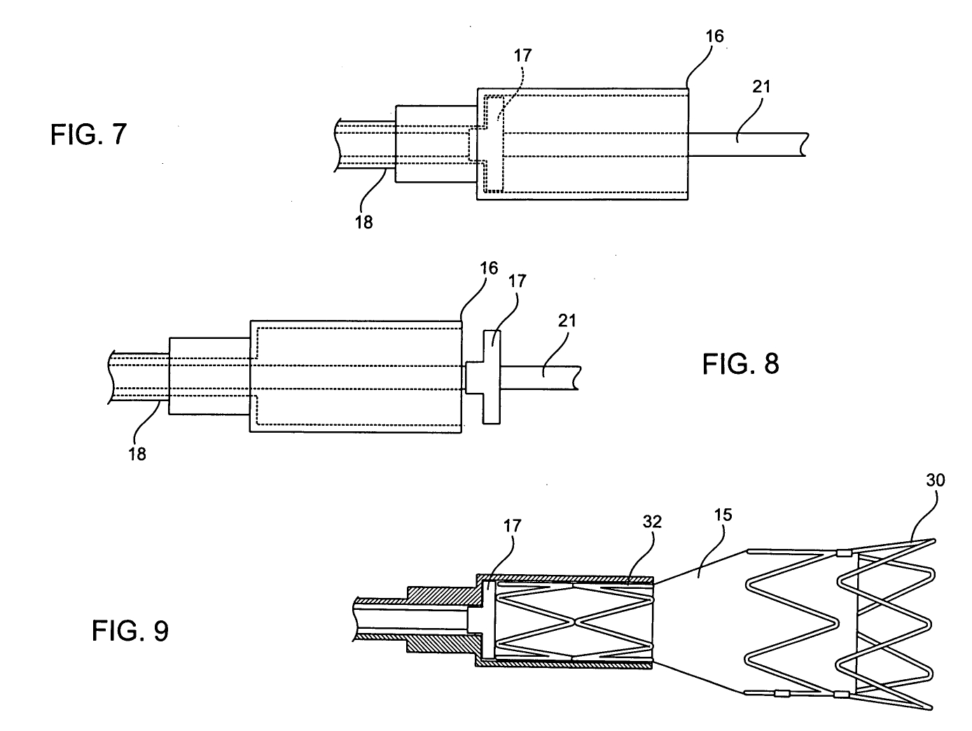

Note that FIGS. 7 and 8 provide a closer view of the relative movement between the release plate 17 which is attached to the catheter 21 and the cup 16. With reference to FIGS. 9 and 10, a closer view is once again shown with the addition of the stent-graft 15 illustrating the proximal apexes or spring 30 fully deployed as well as distal apexes or spring 32 shown in a compressed or constrained arrangement in FIG. 9 before full deployment and the springs 32 shown fully deployed in FIG. 10. Note that the stent-graft 15 may also include radiopaque markers 35 as shown.

Referring to FIG. 11, a flow chart illustrates a method 50 of deploying a stent-graft that includes the step 52 of loading the stent-graft deployment system with a stent-graft, tracking the stent-graft deployment system over a guide wire to a desired location (which may include a curved portion) within a vessel at step 54, and retracting the primary sheath at step 56 which in certain embodiments can expose a secondary sheath that also constrains the stent-graft. The stent-graft can be moved to the desired location while the primary sheath is retracted and the secondary sheath is exposed at optional step 58. The stent-graft is moved to its location within a target area or until its location within the target area is confirmed. It should be noted that once the primary sheath is retracted and the secondary sheath is exposed, the secondary sheath (being of a relatively more flexible material than the primary sheath) will provide greater flexibility in tracking through the remainder of the target area regardless of the curvature or tortuous nature of the vessel. When a secondary sheath is used, the method further includes the step of further tracking the stent-graft deployment system to place the secondary sheath in the curved portion of the target area, and retracting the secondary sheath to at least partially deploy the stent-graft in the target area. This step may include deploying or releasing the stent-graft from the delivery system using a release mechanism such as a release plate. Thus, at step 60, the method preferably axially moves a cup back out from a release plate as previously described. This can be done by rotating a luer or handle (such as handle 26 shown in FIGS. 106) that coaxially moves a catheter coupled to the cup and causes the cup to move relative to the release plate. Once the relative motion of the release plate moves near an edge of the cup, the stent-graft is deployed at step 62.

Embodiments shown are ideally suited for introducing the stent-graft deployment system into a femoral artery and advancing the stent-graft deployment system through an iliac artery into the aorta for repair of an aortic aneurysm and more specifically in tracking the stent-graft deployment system through a portion of an thoracic arch when the secondary sheath has been exposed after the retraction of the primary sheath and without any kinking of the primary sheath. The embodiments shown also provide greater control in the final placement and deployments

Additionally, the description above is intended by way of example only and is not intended to limit the spirit and scope of the invention and it equivalent as understood by persons skilled in the art.

What is claimed is:

1. A stent-graft release mechanism, comprising:
a catheter;
a coaxial inner tube having a cup at a distal end thereof, wherein the coaxial inner tube is placed about the catheter;
a release plate affixed to the catheter; and
a mechanism for axially moving the cup relative to the release plate.

2. The stent-graft release mechanism of claim 1, wherein the mechanism axially moves the release plate relative from a proximal portion of the cup to a position near a distal edge of the cup.

3. The stent-graft release mechanism of claim 1, wherein the cup retains a substantial portion of a spring at a distal end of a stent-graft before deployment.

4. The stent-graft release mechanism of claim 3, wherein the cup retains ends of Nitinol springs at the distal end of the stent graft.

5. The stent-graft release mechanism of claim 1, wherein the mechanism for axially moving is used to deploy the distal end of the stent-graft.

6. The stent-graft release mechanism of claim 1, wherein the mechanism for axially moving the release plate comprises a rotating luer that moves the catheter relative to the cup.

7. The stent-graft release mechanism of claim 1, wherein the cup has a depth of at least 0.25 inches.

8. The stent-graft release mechanism of claim 1, wherein the cup has a depth of at least 40% of the compressed axial length of the distal stent spring of the stent-graft.

9. The stent-graft release mechanism of claim 1, wherein the cup has a depth of at least 50% of the compressed axial length of the distal stent spring of the stent-graft.

10. The stent-graft release mechanism of claim 1, wherein the cup has a depth of at least 66⅔% of the compressed axial length of the distal stent spring of the stent-graft.

11. The stent-graft release mechanism of claim 1, wherein the cup has a depth of at least 100% of the compressed axial length of the distal stent spring of the stent-graft.

12. A stent-graft deployment system, comprising:
a stent-graft;
a catheter having a flexible catheter tip attached to a catheter shaft of the catheter;
a retractable primary sheath containing said stent-graft in a first constrained small diameter configuration around said catheter shaft near said flexible tip;
a cup plunger having a cup operatively coupled at the end thereof for retaining a distal end of the stent graft in a constrained position, wherein the cup plunger moves coaxially in relation to the catheter and the retractable primary sheath; and
a release plate fixed to the catheter, wherein as the cup plunger moves coaxially relative to the release plate the distal end of the stent graft is exposed beyond an outer edge of the cup.

13. The stent-graft deployment system of claim 12, wherein the system further comprises a flexible secondary sheath uncoupled to and disposed within said retractable primary sheath and also containing said stent-graft, wherein when said primary sheath is removed from around said stent-graft, said flexible secondary sheath contains said stent-graft in a second constrained small diameter configuration around said catheter shaft near said flexible tip, wherein removal of the secondary sheath releases the stent-graft from the second constrained small diameter configuration so that stent-graft deployment may proceed using the cup plunger and release plate.

14. The stent-graft deployment system of claim 12, wherein the retractable primary sheath is comprised of a semi-rigid material such as PTFE and the secondary sheath is selected from the group of materials comprising woven materials such as fabrics, porous materials such as ePTFE, polymers such as ultra thin walled polymers, and flexible materials such as PET.

15. The stent-graft deployment system of claim 12, wherein the mechanism for axially moving the release plate comprises a rotating luer that moves the catheter relative to the cup.

16. The stent-graft deployment system of claim 12, wherein the stent-graft deployment system further comprises a retention mechanism for retaining a distal area of the stent-graft in a constrained diameter configuration while remaining within a cap within the flexible catheter tip while still enabling axial and radial movement of the stent-graft.

17. The stent-graft deployment system of claim 12, wherein the cup has a depth of at least 0.25 inches.

18. The stent-graft deployment system of claim 12, wherein the cup has a depth ranging from about 25% of a longitudinal length of a spring at the distal end of the stent-graft up to 100% of the length of the spring.

19. A method of deploying a stent-graft using a stent-graft deployment system having a stent-graft release mechanism and a retractable primary sheath, comprises the steps of:
loading the stent-graft deployment system with a stent-graft, wherein the distal end of the stent-graft is retained within a cup of the stent-graft release mechanism;
tracking the stent-graft deployment system over a guide wire to a desired location within a vessel;
retracting the primary sheath to expose at least a proximal portion of the stent-graft; and
moving said cup from a position where a release plate is located within a lower portion of the cup to a position where the release place is located beyond a distal edge of the cup to at least partially deploy the stent-graft in the target area.

20. The method of claim 19, wherein the method further comprises the step of retaining apexes of Nitinol springs of the distal end of the stent-graft within the cup before deployment.

21. The method of claim 19, wherein a catheter is coupled to the release plate and wherein the step of moving the release plate comprises the step of rotating a luer that coaxially moves the catheter relative to the cup.

22. The method of claim 19, wherein the stent-graft deployment system further comprises a secondary sheath within the primary sheath and wherein the method further comprises the step of moving the stent-graft to a location within the target area while the primary sheath is retracted as the secondary sheath is exposed.

23. The method of claim 19, wherein the step of moving the release plate comprises the step of axially moving the release plate relative to the cup.

24. The method of claim 19, wherein the method further comprises the step of releasing the stent-graft from the delivery system after moving the cup to a location where the location of the release plate is beyond an edge of the cup.
